(19) United States
(12) Patent Application Publication
(10) Pub. No.: US 2003/0045894 A1
(43) Pub. Date: Mar. 6, 2003

(54) Method of using a cornea cut to track eye movement during laser vision correction surgery

(76) Inventor: Jack T. Holladay, Bellaire, TX (US)

Correspondence Address: MANELLI DENISON & SELTER PLLC, 7th Floor, 2000 M Street, N.W., Washington, DC, 20036-3307, US

(21) Appl. No.: 09/946,585

(22) Filed: Sep. 6, 2001

(51) Int. Cl. A61F 9/00

(52) U.S. Cl. 606/166

(57) ABSTRACT

A method of tracking an eye during vision correction treatment includes cutting corneal tissue to define a cut edge. At least a portion of the cut edge is tracked to track eye movements. In one embodiment, the corneal tissue is cut to define a flap-like layer such that fluid gathers near the cut edge. The fluid is illuminated prior to tracking the cut edge.

### FIELD OF THE INVENTION

The invention generally relates to tracking eye movements during laser vision correction surgery.

### BACKGROUND OF THE INVENTION

One of the most significant challenges refractive surgeons face today is eye movement during laser vision correction surgery. Although in many cases patient fixation on the light source is adequate, voluntary visual fixtation cannot eliminate all eye movements. In fact, during fixation, saccadic square wave jerks occur within a range of 0.5 to 3 degrees. With the advent of custom ablations and the need for aspheric surfaces, compensating for even the slightest eye movements is imperative.

One solution is to manually fixate the globe during surgery. This technique has met with some success, but it increases the risk for off-axis treatments, especially with changes in head position during the ablation procedure. Furthermore, manual fixation will not compensate for head movement induced by patients with a strong heartbeat, nor will it compensate for patient movement during surgery.

The use of an eye tracker is the method of choice for following eye movements during laser surgery. Advanced eye trackers not only track eye movements, they also adjust laser pulses to ensure that the laser is producing the appropriate ablation pattern at the desired position on the cornea.

Two types of eye trackers are in current use: infrared (IR) video-based trackers and laser trackers. The IR video-based tracker employs a video camera to track the pupil using reflected IR light. The full image of the pupil is acquired and transferred to an image-processing unit, which digitizes and processes the incoming information. This tracking technique is the most common method of eye tracking because it enhances accuracy and is relatively easy to use. No pupil dilation is necessary, and the video-based system can track varying pupil sizes. Even with small pupils, typically 250 data points are obtained for each image at the pupillary border for accurate pupil positioning.

Laser trackers measure reflected IR light from the iris-pupil boundaries using sensitive detectors. The laser tracker identifies four points on the pupil-iris border and calculates the center of the pupil by circular interpolation through this reflection technique. To obtain sufficient accuracy using laser trackers, the pupil needs to be significantly large and of a fixed diameter. To accomplish this, the patient's pupil needs to be dilated to 6.5 mm or greater. Although the four pupillary points may be obtained with laser trackers at higher rates than with video-based trackers, the obtained laser tracker data must be filtered to obtain the same degree of accuracy. The resultant speed of laser trackers is similar to that of standard video trackers. However, video trackers permit a more robust detection and elimination of surgical artifacts.

In employing either of the above-described eye tracking methods, errors in tracking can occur in due to parallax, since the pupil being tracked is not in the same plane as tissue being treated. Thus, there is a need to provide a method of tracking the eye during laser vision correction surgery such that the surface of the eye being tracked is in the same plane as tissue being treated.

### SUMMARY OF THE INVENTION

An object of the invention is to fulfill the need referred to above. In accordance with the principles of the present invention, this objective is achieved by providing a method of tracking an eye during vision correction treatment. The method includes cutting corneal tissue to define a cut edge. A portion of the cut edge is tracked to accommodate for eye movements. In one embodiment, the corneal tissue is cut to define a flap-like layer such that fluid gathers near the cut edge. The fluid is illuminated prior to tracking the cut edge.

In accordance with another aspect of the invention, a method of tracking an eye during vision correction treatment includes cutting the cornea to define a flap-like layer having a hinged portion keeping the flap-like layer attached to the cornea. The flap-like layer is moved about the hinged portion to expose a stromal bed surface. At least a portion of the hinged portion is tracked. The hinge can also be tracked to provide torsional motion of the eye.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, wherein like reference numerals refer to like parts, in which:

FIG. 1 is a sectional view of an eye shown with a portion of the cornea being cut to define a flap-like layer.

FIG. 2 is a plan view of the eye of FIG. 1.

FIG. 3 is a perspective view of a conventional system for illuminating and scanning the eye during vision correction treatment, shown with a camera near an eye being treated.

FIG. 4 is a perspective view of a conventional system for illuminating and scanning the eye during vision correction treatment, shown with a camera disposed above an eye being treated.

FIG. 5 is a sectional view of an eye shown with a portion of the cornea being removed.

FIG. 6 is a plan view of the eye of FIG. 5.

DETAILED DESCRIPTION OF THE PREFERRED EMBODIMENT

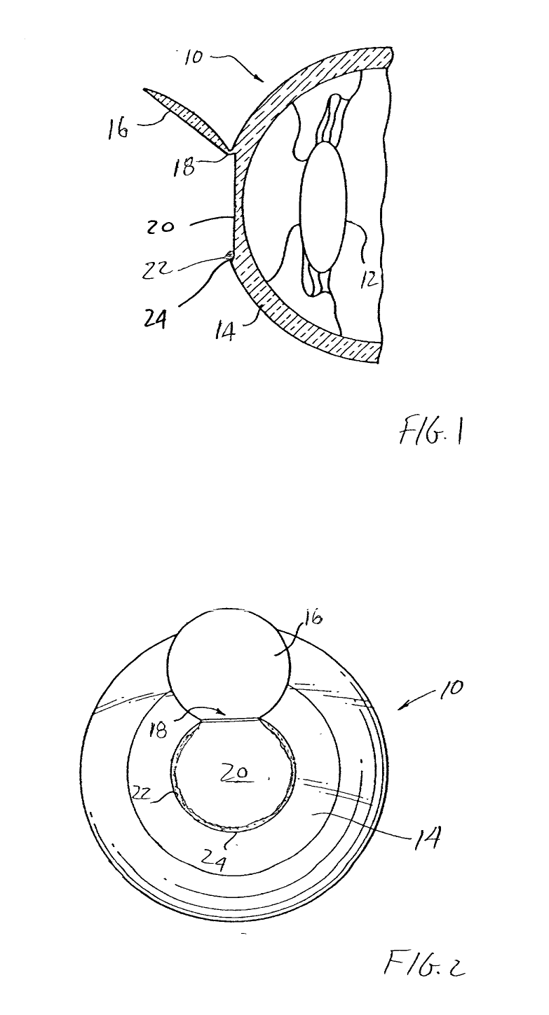

With reference to FIG. 1, a cross-sectional view of an eye 10 is shown which includes a lens 12 and cornea 14. In the conventional manner, a flap-like layer 16 of the cornea 14 is formed by separating the flap-like layer 16 from the cornea 14 with a cutting tool, such as a microkeratome, excimer (e.g., ultraviolet) laser, infrared (e.g., Er:YAG) laser, or the like. The flap-like layer 16 is typically about 160-175 microns in thickness and includes the epithelium and Bowman layer. The flap-like layer 16 remains attached to the cornea 14 by a hinged portion 18. When the flap-like layer 16 is folded back or pivoted about the hinged portion 18, the stromal bed surface 20 is exposed for vision correction treatment.

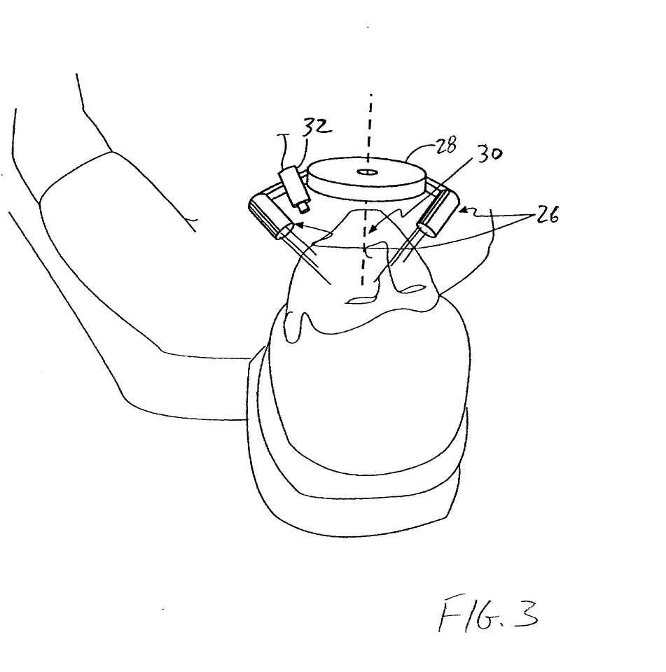

With reference to FIG. 2, in creating the flap-like layer 16, fluid 22 gathers near the cut edge 24. Instead of tracking the pupil, as in the conventional manner, in accordance with the disclosed embodiment, at least a portion of the edge 24 and/or the hinged portion 18 is tracked. Due to the meniscus of the fluid 22 collecting near the edge 24, when illuminated, the edge 24 is easy to detect with conventional tracking equipment. Also, at least a portion of the hinged portion 18 can be recognized and tracked.

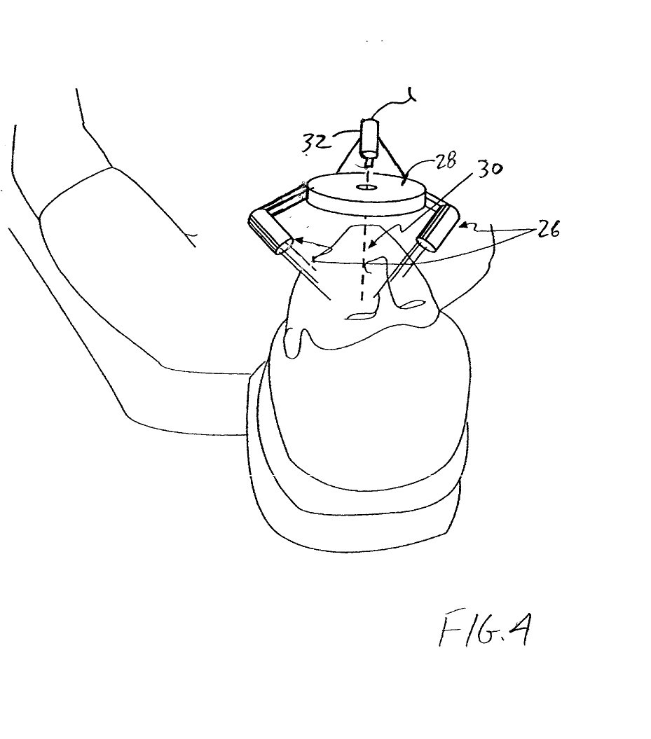

FIG. 3 shows a conventional eye tracking system which can be used in tracking the edge 24 and/or the hinge portion 18 of the cornea 14 during laser vision correction surgery. The conventional illumination system includes one or two infrared light bundles 26, mounted on a central hub 28, to illuminate the edge 24 and/or the hinged portion 18 for tracking by an eye tracking system. A path of a visible light beam used during vision correction treatments is shown at 30. A camera 32, sensitive to IR illuminations and fixed with respect to the subject's head, scans at least a portion of the edge 24 and/or hinged portion 18 to provide a video image for tracking the position of the eye. As shown in FIG. 4, the camera 32 can be disposed on the same axis as the visible light 30. An example of eye tracking systems that can be employed in the tracking method of the embodiment are the VOG eye tracking systems commercially available from SensoMotoric Instruments (SMI) of Berlin, Germany. It can be appreciated that other conventional techniques used to track the pupil can be modified and employed to track the edge 24 and/or the hinged portion 18. Furthermore, visible and infrared light sources can be used for illumination during tracking.

In certain situations, laser vision treatment can fall outside the stromal bed surface 20, or may fall on the edge 24. However, since the tracking method of the disclosed embodiment can detect the edge 24, electronic masking techniques can be employed to prevent treatment on the edge 24 or in the epithelium outside of the edge 24.

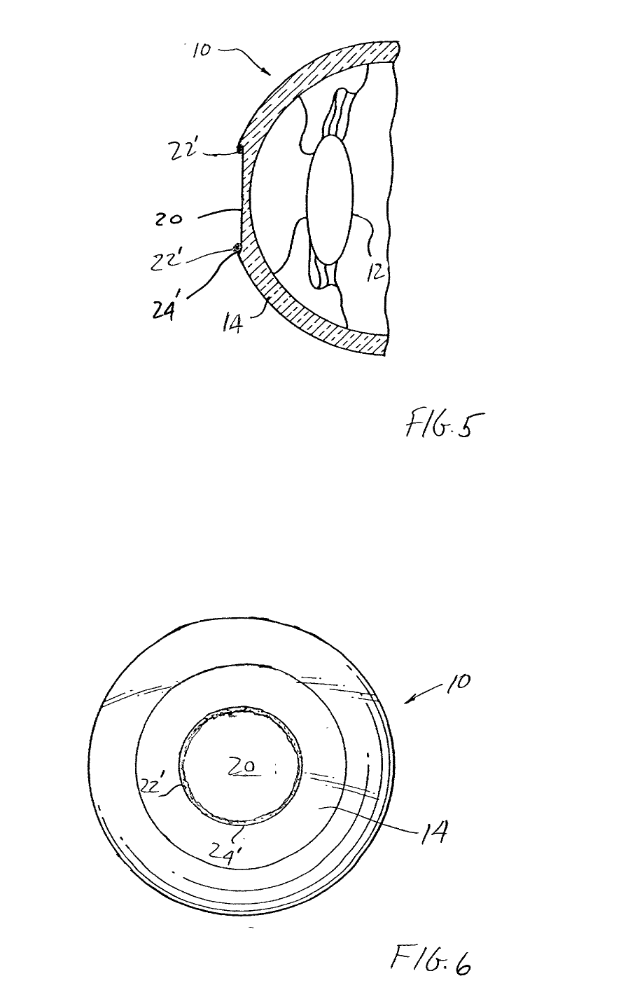

The method of the invention can be employed with another LASIK procedure wherein the flap-like layer 16 or cap is removed completely from the cornea 14. In this procedure, as shown in FIGS. 5 and 6, the cut created to remove the cap and expose a generally circular bed 20 defines the edge 24′. In the manner discussed above with regard to the embodiment of FIGS. 1 and 2, fluid gathers near the edge 24′ thus making the edge 24′ easy to track. Once the corrective procedure is completed, the cap is replaced on the cornea 14.

The inventive eye tracking method is also applicable to a technique called surface Photorefractive Keratectomy (PRK) whereby the epithelium is removed with a laser, scraper, or brush. Thus, no flap-like layer remains. Removal of the epithelium is referred to herein as “cutting” but, it is understood that, “cutting” includes any method of removing the epithelium. The cut to remove the epithelium is less deep than the LASIK cut discussed above, however, the cut does define an edge where fluid collects. Thus, the edge is easy to track in this procedure as well.

Furthermore, the hinge can be tracked to provide torsional motion of the eye.

Hence, the disclosed method of tracking the eye during laser vision correction surgery eliminates errors caused by parallax since the surface of the eye being tracked is in the same plane as tissue being treated.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

1. A method of tracking an eye during vision correction treatment, comprising:
cutting corneal tissue to define a cut edge; and
tracking at least a portion of the cut edge.

2. The method of claim 1, wherein the cutting step comprises:
cutting the corneal tissue to define a flap portion such that fluid gathers near the cut edge; and
moving the flap portion about a hinged portion to expose a stromal bed surface.

3. The method of claim 1, wherein the cutting corneal tissue step comprises:
cutting the corneal tissue; and
completely removing said cut corneal tissue such that fluid gathers near the cut edge.

4. The method of claim 3, wherein the cutting the corneal tissue step comprises:
cutting epithelium tissue.

5. The method of claim 2, further comprising:
illuminating the fluid to track the cut edge.

6. The method of claim 3, further comprising:
illuminating the fluid to track the cut edge.

7. The method of claim 1, wherein the cutting corneal tissue step comprises:
cutting the corneal tissue with a keratome.

8. The method of claim 1, further comprising:
using a camera to scan the portion of the cut edge to provide a video image for tracking a position of the eye.

9. The method of claim 1, further comprising:
treating a stromal bed surface for vision correction.

10. A method of detecting a portion of a cornea during vision correction treatment, the method comprising:
cutting corneal tissue to define a cut edge; and
detecting a position of the cut edge.

11. The method of claim 10, further comprising:
preventing laser ablation in corneal tissue on the cut edge.

12. The method of claim 11, wherein the preventing laser ablation step includes:
defining an electronic mask.

13. The method of claim 10, further comprising:
preventing laser ablation in corneal tissue outside of the cut edge.

14. The method of claim 11, wherein the preventing laser ablation step includes:
defining an electronic mask.

15. A method of tracking an eye during vision correction treatment comprising:
cutting a cornea to define a flap portion having a hinged portion keeping the flap portion attached to the remainder of the cornea,
moving the flap portion about the hinged portion to expose a stromal bed surface; and
tracking at least a portion of the hinged portion.

16. The method of claim 15, wherein the step of cutting a cornea comprises:
cutting the cornea with a keratome.

17. The method of claim 15, further comprising:
using a camera to scan the portion of hinged portion to provide a video image to track a position of the eye.

18. The method of claim 15, further comprising:
treating the stromal bed surface for vision correction.

19. The method of claim 15, wherein the step of tracking at least a portion of the hinge portion includes:
obtaining information relating to torsional motion of the eye.

20. A system for tracking an eye during vision correction treatment, the system comprising:
means for cutting corneal tissue to define a cut edge; and
means for tracking at least a portion of the cut edge.

21. The system of claim 20, wherein the means for cutting comprises:
a keratome.

22. The system of claim 20, wherein the means for tracking includes:
means for illuminating fluid gathering near the cut edge to track the cut edge.

23. The system of claim 20, further wherein the means for tracking includes:
a camera to scan the portion of the cut edge to provide a video image for tracking a position of the eye.

24. A system for detecting a portion of a cornea during vision correction treatment, the system comprising:
means for cutting corneal tissue to define a cut edge; and
means for detecting a position of the cut edge.

25. The system of claim 24, further comprising:
means for preventing laser ablation of the cut edge.

26. The system of claim 25, wherein the means for preventing ablation of the cut edge comprises:
an electronic mask.

27. The system of claim 24, further comprising:
means for preventing laser ablation in corneal tissue outside of the cut edge.

28. The system of claim 27, wherein the means for preventing ablation in corneal tissue outside of the cut edge comprises:
an electronic mask.

29. A system for tracking an eye during vision correction treatment comprising:
means for cutting a cornea to define a flap portion having a hinged portion keeping the flap portion attached to the remainder of the cornea; and
means for tracking at least a portion of the hinged portion.

30. The system of claim 29, wherein the means for cutting a cornea comprises:
a keratome.

31. The system of claim 29, wherein the means for tracking includes:
a camera to scan the portion of hinged portion to provide a video image to track a position of the eye.

32. A method of tracking an eye during vision correction treatment, comprising:
identifying a cut edge in corneal tissue; and
tracking at least a portion of the cut edge.

33. A method of tracking an eye during vision correction treatment, comprising:
identifying a cut edge in corneal tissue; and
tracking at least a portion of the cut edge.
